(19) United States
(12) Patent Application Publication
(10) Pub. No.: US 2004/0142322 A1
(43) Pub. Date: Jul. 22, 2004

(54) Continuous non-radioactive polymerase assay

(75) Inventors: Bruce A. Malcolm, Westfield, NJ (US); Frederick C. Lahser, Springfield, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/679,987

(22) Filed: Oct. 7, 2003

Related U.S. Application Data
(60) Provisional application No. 60/416,760, filed on Oct. 7, 2002.
(60) Provisional application No. 60/487,388, filed on Jul. 15, 2003.

(51) Int. Cl. C12Q 1/70; C12Q 1/68; C12P 19/34

(52) U.S. Cl. 435/5; 435/6; 435/91.2

(57) ABSTRACT

This invention provides a sensitive method for detecting RNA-dependent polymerase activity and for characterizing inhibitors of these enzymes, particularly NS5B of HCV and RT of HIV. The invention further provides methods wherein the elongation of RNA is measured in real time through the continuous detection of light generated from a reaction cascade involving a coupled enzyme system based on firefly luciferase.

This application claims benefit of priority from U.S. Provisional Applications 60/416,760, filed Oct. 7, 2002 and 60/487,388, filed Jul. 15, 2003.

### BACKGROUND OF THE INVENTION

In the last two decades, a number of RNA viruses have emerged to become increasing threats to human health, including Human Immunodeficiency virus (HIV), Ebola virus, Hantavirus Pulmonary Syndrome (Four Corners virus), Hepatitis C virus (HCV), Dengue virus, Yellow Fever virus, West Nile virus and, most recently, Severe Acute Respiratory Sundrome (SARS). These threats provide an increasing awareness that viral predators still present a constant threat to individual survival as well as to the success of the population at large.

According to the Joint United Nations Programme on HIV/AIDs, as of the end of 2001, 40 million people were estimated to be living with HIV/AIDS. Of these, 37.2 million were adults, 17.6 million were women, and 2.7 million were children under age 15. In the course of 2001, AIDS caused the deaths of an estimated 3 million people, including 1.1 million women and 580,000 children under 15.

In addition to the problem of HIV, HCV is now recognized as the causative agent for most cases of non-A and non-B hepatitis, with an estimated human seroprevalence of 1% globally. See Choo, et al., Science, 244:359-362 (1989); Kuo, et al., Science, 244:362-364 (1989); Purcell, FEMS Microbiology Reviews, 14:181-192 (1994). In the United States alone, four million individuals may be infected with HCV. See Alter and Mast, Gastroenterol. Clin. North Am., 23:437-455 (1994).

Unfortunately, a salient characteristic of RNA viruses is that they have relatively high rates of spontaneous mutation reportedly on the order of 10⁻³ to 10⁻⁴ per incorporated nucleotide. See Fields & Knipe (1986) “Fundamental Virology” (Raven Press, NY). As a result, primary isolates of RNA viruses such as HIV and HCV display rapid immune evasion, thus making vaccine development exceedingly difficult, expensive and unsuccesful so far.

In the absence of effective vaccines to prevent infection, identification of novel therapeutics that block or inhibit such specific viral enzymes as polymerases and proteases are becoming a key step in slowing the disease process caused by these RNA viruses. For example, existing therapies using alpha interferon alone and the combination of alpha interferon and ribavirin are effective, but only in a portion of patients having chronic HCV infection. Therefore, the development of additional small molecule inhibitors against viral enzymes has become critical. This process is aided in the area of anti-HCV research due to the completion of crystal structures for the HCV protease (R. A. Love et al., Cell 87:331-342 (1996)); HCV NS3 RNA helicase (N. Yao et al., Nat. Struct. Biol. 4:463-467 (1997)); and the HCV NS5B RNA-dependent RNA-polymerase (C. A. Lesburg et al., Nat. Struct. Biol. 6:937-943 (1999)).

Since HCV is a positive-strand RNA virus, the NS5B RNA-dependent RNA polymerase (RdRp) provides an especially attractive target for developing inhibitors, because this enzyme is vital for genome replication and there is no human counterpart. Inhibitors of viral enzymes that have no human analogs are particularly advantageous as therapeutics because they may display reduced side effects. In HCV, genome replication proceeds in two steps: synthesis of complementary minus-strand RNA using the genome as template and the subsequent synthesis of genomic RNA using this minus-strand RNA as template. The key enzyme involved in both of these steps is the virally encoded RdRp.

The development of an effective enzyme inhibitor against RdRp requires a sensitive assay for detecting enzyme inhibition and for characterizing the inhibitor. The current assays for RdRp are inadequate for efficiently discovering and characterizing new inhibitors. For example, one disadvantage of the existing assays for HCV NS5B RdRp activity is that they are based on the detection of radiolabeled RNA products that are synthesized during primer-dependent elongation from radiolabeled nucleotide triphosphates (NTPs). (Lohmann et al., J. Viral Hepat. 7:167-174 (2000); Reigadas et al., Eur. J. Biochem. 268:5857-5867 (2001); Zhong et al., J. Virol. 74:2017-2022 (2000) and U.S. Pat. No. 6,383,768 to DeFrancesco et al., (2002). Most of these assays are gel-based, in which radiolabeled RNAs are visualized after gel electrophoresis, but variations exist where extension products are detected by scintillation-proximity methods [Ferrari et al., J. Virol. 73:1649-1654 (1999)]. In the scintillation-proximity assay (SPA), a biotinylated primer (often a 12-base oligonucleotide of G) is annealed to a homopolymer (e.g., a poly(C)) to establish a template for extension from the primer by the polymerase in the presence of unlabeled and radiolabeled nucleotide (GTP and [³H]-GTP). Terminated reactions are subsequently analyzed by the capture of the radiolabeled and biotinylated product on streptavidin beads, and detection of radioactivity by scintillation counting.

Another disadvantage of many existing assays is that the results are obtained only after radiolabeled RNAs are separated by gel electrophoresis. Although radioactive signals are easily detected and quantified, gel electrophoresis based radiolabeled detection procedures is time consuming, cumbersome and expensive because the gel must be dried, film must be exposed, developed and oftentimes analyzed on an additional reader to be quantified. All of these steps require special experimental handling and waste processing because of the radioactive by-products generated. Another disadvantage of existing assay systems is that they inherently produce end-point measurements. It is possible to produce kinetic data with these assays, but the processing of multiple time-point samples for each experimental variation, such as time and drug concentration, becomes progressively more tedious and increases the amount of radioactivity for disposal.

In addition to the assays involving radiolabeled nucleotides, there are two non-radioactive calorimetric polymerase assay systems available that may be of use with RdRp. The first calorimetric assay for RNA polymerase is dependent on the incorporation of ATP or GTP with a p-nitrophenyl moiety attached to the gamma (γ)-phosphate (PNP-NTP). In this system, the PNP-NTP is incorporated during the polymerization reaction and releases a PNP-pyrophosphate (PNP-PPᵢ) as a by-product of the reaction. The PNP-PPᵢ by-product is subsequently hydrolyzed by calf intestinal phosphate (CIP) to produce the chromophore p-nitrophenylate, which is detectable at 405 nm. The authors demonstrated that the Brome Mosaic virus (BMV), Bovine Viral Diarrhea virus (BVDV) and T7 RNA polymerase were able to use the modified NTP, however each viral enzyme is different and may or may not accept these altered substrates. See Vassiliou et al., Virology 274:429-437 (2000).

A second non-radioactive assay for RdRp has been described in which an RNA template is covalently attached to a solid surface. See Park et al., J. Virol. Methods 101:211-214 (2002). In this endpoint assay, the polymerase reactions were initiated on the RNA template in the presence of biotin-16-UTP. The polymerase reactions were then terminated and the products were quantified for incorporated biotin at an endpoint. The quantification step involves adding a streptavidin-conjugated alkaline phosphatase and a phosphatase substrate such as para-nitrophenolphosphate, then allowing the phosphatase reaction to proceed to produce a chromophore detectable at 405 nm. The development of the chromophore may be followed using a spectrophotometer. This assay is not particularly amenable to obtaining kinetic data, and obtaining accurate values for the kinetic constants of the reaction may be compromised by the high concentrations of reagents necessary for product detection by calorimetric means. More importantly, the use of enzyme and substrate at these levels may confound analyses of inhibitors due to their concentrations relative to the Km of the assay components.

Therefore, in view of the state of the art, a need exists for broadly applicable assays of RdRp activity and other RNA-dependent polymerase activity, where preferably no radioactive waste is generated and where the reaction components can be used at low concentrations. A further need exists for an assay with a broad dynamic range of detection and where the data can be collected in a real time continuous format that provides relevant information about the kinetic parameters of the enzyme inhibitors with reasonably high throughput.

### SUMMARY OF THE INVENTION

The present inventors have fulfilled the above-identified needs by providing methods for detecting RNA-dependent polymerase activity and for characterizing inhibitors of RNA-dependent polymerases that do not use radioactivity and can provide data in a real time continuous manner. These methods may be used for detecting and/or measuring RNA-dependent RNA polymerase activity or RNA-dependent DNA polymerase activity.

In a preferred embodiment, this invention provides a sensitive method for detecting RNA-dependent RNA polymerase (RdRp) activity and for characterizing inhibitors of these enzymes, particularly NS5B of HCV. The invention further provides methods wherein the elongation of RNA is measured continuously in real time through detection of light generated from a reaction cascade involving a coupled enzyme system based on firefly luciferase. Specifically, this invention provides methods for detecting RdRp activity using a modified version of the reaction cascade used in the non-radioactive sequencing technology known as PYROSEQUENCING™. See Nyren et al., Anal. Biochem. 151(2), 504-9(1985); Nyren, Anal. Biochem. 167:235-238 (1987); Ronaghi et al., Anal. Biochem. 242:84-89 (1996).

In another embodiment, the invention provides a method for detecting RNA-dependent DNA polymerase activity and for characterizing inhibitors of these enzymes, particularly for the reverse transcriptase of HIV.

In one embodiment, the present invention provides a method for detecting RNA-dependent polymerase activity comprising: (a) providing a primer oligonucleotide having a 3′ OH; (b) contacting said primer oligonucleotide with a template polynucleotide and allowing hybridization to occur to form a hybridized polynucleotide; (c) combining said hybridized polynucleotide with an RNA-dependent polymerase or an enzyme mixture comprising said RNA-dependent polymerase, wherein apyrase is not part of the enzyme mixture; (d) adding a PPᵢ detection mixture to said enzyme mixture; (e) initiating a polymerization reaction by the addition of a substrate mixture comprising a nucleotide triphosphate or an analog thereof; and (f) measuring a product of the PPᵢ detection mixture. In a preferred embodiment, the PPᵢ detection mixture comprises luciferase, luciferin, ATP sulphurylase and adenosine 5′-phosphosulfate (APS) and the measuring step measures the amount of light emitted. Although adding the PPᵢ detection mixture is shown as a separate step in the method, it may be added with the RNA-dependent polymerase or the enzyme mixture comprising said polymerase. Further, the PPᵢ detection mixture may be added before addition of the RNA-dependent polymerase or enzyme mixture comprising said polymerase, or may be added after initiation of the polymerization reaction.

This invention also provides a method for evaluating or characterizing inhibitors of an RNA-dependent polymerase comprising: (a) providing a primer oligonucleotide having a 3′ OH; (b) contacting the primer oligonucleotide with a template polynucleotide and allowing hybridization to occur to form a hybridized polynucleotide; (c) combining said hybridized polynucleotide with an RNA-dependent polymerase or an enzyme mixture comprising said RNA-dependent polymerase, wherein apyrase is not part of the enzyme mixture; (d) adding a PPᵢ detection mixture to said enzyme mixture; (e) initiating a polymerization reaction by the addition of a substrate mixture comprising a nucleotide triphosphate or an analog thereof and an inhibitor; (f) measuring a product of the PPᵢ detection mixture. In a preferred embodiment, the PPᵢ detection mixture comprises luciferase, luciferin, ATP sulphurylase and adenosine 5′-phosphosulfate (APS) and the measuring step measures the amount of light emitted. In certain embodiments, the inhibitor may be either pre-combined with the RNA-dependent polymerase or combined with the substrate mixture. Further, although adding the PPᵢ detection mixture is shown as a separate step in the method, it may be added with the RNA-dependent polymerase or the enzyme mixture comprising said polymerase. Further, the PPᵢ detection mixture may be added before addition of the RNA-dependent polymerase or enzyme mixture comprising said polymerase, or may be added after initiation of the polymerization reaction.

In another embodiment, the RNA-dependent polymerase is a viral polymerase selected from the group of viruses consisting of hepatitis C virus, poliovirus, West Nile virus, Dengue virus, Human Immunodeficiency virus, Human T Cell Leukemia virus, St. Louis Encephalitis virus, Yellow Fever virus and Measles virus. In a preferred embodiment, the viral polymerase is an RNA-dependent RNA polymerase (NS5B) from Hepatitis C virus. In another embodiment, the viral polymerase is an RNA-dependent RNA polymerase from poliovirus (3D polymerase; 3D^{pol}). In another preferred embodiment, the viral polymerase is an RNA-dependent DNA polymerase known as reverse transcriptase (RT) from HIV (HIV RT). The HIV may be Type I (RT-I) or Type II (RT-II). The polymerase may be recombinantly produced or obtained from natural sources. The polymerase may be substantially purified or enriched by various means known in the art to purify or enrich proteins.

In another embodiment of the present invention, the hybridized polynucleotide comprises synthetic poly(A) and poly(U). The template polynucleotide may be either poly(A) or poly(U) and the primer would be the corresponding complementary oligonucleotide, i.e., oligo(U) or oligo(A) or an analog of A.

In another embodiment of the present invention, the hybridized polynucleotide comprises synthetic poly(G) and poly(C). The template polynucleotide may be either poly(G) or poly(C) and the primer would be the corresponding complementary oligonucleotide i.e., oligo(C) or oligo(G), respectively. In another embodiment of the present invention the template is a combination polymer of poly(A,G,C). The polynucleotide may be in certain specific proportions such as ⅓ A, ⅓ G and ⅓ C or any other combination of nucleotides including random combinations.

In one embodiment, the primer polynucleotide and template polynucleotide are on separate molecules. In another embodiment of the invention, the primer polynucleotide and the template polynucleotide are on the same RNA molecule. The hybridized polynucleotide may be formed though a fold back internal hybridization of the primer polynucleotide to the template polynucleotide, which are on the same RNA molecule. In a further embodiment, the hybridized polynucleotide is formed though a fold back internal hybridization of the primer sequence, having a 3′-OH, oligonucleotide to the template polynucleotide, which are on the same RNA molecule, forming a defined hairpin RNA structure.

In one embodiment of the invention, the enzyme mixture comprises luciferase, ATP sulfurylase, and an RNA-dependent polymerase such as HCV NS5B, poliovirus 3D polymerase (PV 3D^{pol}) or HIV RT.

In a certain embodiment of the invention, the substrate mixture comprises adenosine 5′-phosphosulfate and D-luciferin. In another embodiment of the invention, the substrate mixture further comprises one or more of the following ribonucleotides (NTPs, e.g., for an RdRp) or deoxyribonucleotides (dNTPs, e.g., for an RNA-dependent DNA polymerase (RdDp)): GTP or dGTP, CTP or dCTP, TTP or dTTP, and UTP. Generally, use of ATP is avoided, but derivatives or analogs of ATP or dATP can be used as a substrate. See, e.g., Example 5, which exemplifies one such ATP analog.

In a preferred embodiment of the present invention, the light emitted as a result of the PPᵢ detection reactions following RNA-dependent polymerase activity is measured with a luminometer.

### BRIEF DESCRIPTION OF THE FIGURES

FIG. 1. Linearity of reaction in relation to NS5B concentration. Polymerase was serially diluted and assayed at room temperature in the presence of 1 μM GTP and the oligo(G)₁₂/poly(C) template in a 96-well plate with a final reaction volume of 100 μl. Light was captured for 0.2 seconds (s) every 60 s during a 30 minute reaction.

FIG. 2. Dependence of the coupled reaction on primer. Reactions were set up using a fixed concentration of NS5B (20 nM) and GTP substrate (1 μM), while varying the amount of oligo(G)₁₂ primer used to anneal to the poly(C) template (5 μg/ml) to give the hybridized polynucleotide poly(C)/oligo(G)₁₂. Primer concentrations used in the poly(C)/oligo(G)₁₂ reaction were 0 μg/ml, 0.15 μg/ml, 0.5 μg/ml, 1.5 μg/ml, and 5 μg/ml. Light was captured for 0.1 s every 60 s during a 30 minute reaction.

FIG. 3. Inhibition of HCV NS5B by 3′-deoxyguanosine-5′ triphosphate. Reactions were set using a fixed concentration of NS5B (20 nM) and GTP substrate (1 μM), while varying the inhibitor (0.1-2.5 μM). Light was captured for 0.2 s every 40 s during a 30 minute reaction.

FIGS. 4a-4d. Characterization of PV 3D^{pol} using a continuous assay format. FIG. 4a: Titration of 3D^{pol}. PV 3D^{pol} was serially diluted in storage buffer and assayed (n=2) at room temperature with 1 μM GTP, 0.03U ATP sulfurylase, and the poly(C)/oligo(G)₁₂ (0.5 μg/ml oligo(G)₁₂; 5 μg/ml poly(C)) in a 96-well plate with a final reaction volume of 100 μl. Light was captured for 0.2 s every 45 s during a 30 minute reaction. FIG. 4b: Linearity of reaction with RdRp concentration. Initial velocity estimates from the data in FIG. 4a were plotted against the concentration of 3D^{pol}. FIG. 4c: Effect of primer on activity. Various concentrations of oligo(G)₁₂ primer were added to 5 μg/ml poly(C), with other conditions and analysis parameters as described for FIG. 4a. FIG. 4d: Linearity of reaction with primer concentration. Initial velocity estimates from data in FIG. 4c were plotted against the concentration of oligo(G)₁₂.

FIGS. 5a-5b. Assay using a heteropolymer RNA template and 3D^{pol}. FIG. 5a: Predicted secondary structure of the 75-nucleotide long heteropolymer RNA template, D-RNA, as predicted by Mfold. See Zuker, M. Nucl. Acids Res. 31:1-10 (2003). The annealing site for a complementary 12-mer primer is shown (black arrow). FIG. 5b: Primer dependence of 3D^{pol} on a heteropolymer RNA template. Duplicate reactions contained 60 nM 3D^{pol}, 100 nM each of D-RNA and 12-mer primer (100 nM template RNA), 1 μM of each nucleotide (GTP, CTP, UTP, and ATPαS, and 0.03U ATP sulfurylase. 3D^{pol} and RNA were co-incubated at room temperature for 15 minutes prior to the addition of NTPs to initiate the reactions.

### DETAILED DESCRIPTION

The present invention is based on the detection of free pyrophosphate (PPᵢ), a by-product of the polymerase-mediated nucleotide incorporation reaction. The PPᵢ is converted to ATP by a second reaction containing adenosine 5′-phosphosulfate (APS) and catalyzed by ATP sulfurylase. This ATP pool provides the energy for a luciferase-catalyzed reaction, producing photons in direct proportion to the amount of PPᵢ generated by the viral polymerase. The chemical formulas describing these steps are as follows:

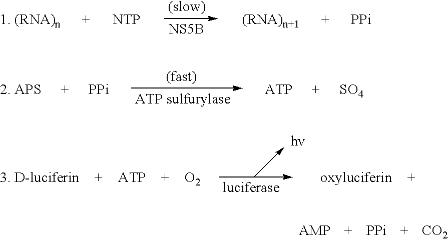

NS5B is used as an example of an RdRp. However, following the teachings of the specification, one skilled in the art would recognize that RdRps other than NS5B can be used in the reaction. Further, one may also use an RNA-dependent DNA polymerase, such as HIV RT, in the reaction. In that case, step 1 would be described by the following formula:

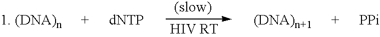

Steps 2 and 3 are the same as those described above.

The continuous RNA-dependent polymerase assay described here differs from the PYROSEQUENCING™ for DNA templates described by Nyren and colleagues. See Nyren, Anal. Biochem. 167:235-238 (1987); Nyren et al., Anal. Biochem. 244:367-373 (1997); Ronaghi et al. Anal. Biochem. 242:84-89 (1996); U.S. Pat. No. 6,258,568 to Nyren (2001) and U.S. Pat. No. 6,210,981 to Nyren et al. (2001). Compared to the PYROSEQUENCING™ method, the template used for extension in the continuous assay of the present invention is RNA. In addition, there is a fundamental difference between the PYROSEQUENCING™ reaction cascade and the reaction cascade of the present invention. The PYROSEQUENCING™ reactions are shown below:

PYROSEQUENCING™ Reactions:

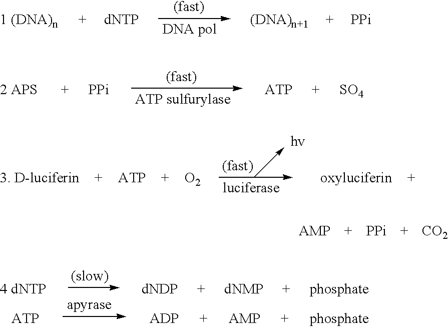

To successfully perform PYROSEQUENCING™, the reaction must be stopped after each nucleotide addition to distinguish nucleotide incorporation from no addition; this provides a specific and strong signal over background for an elongation event. To achieve this, the PYROSEQUENCING™ reaction is intentionally limited by the addition of a single dNTP in a determined order. The PYROSEQUENCING™ enzyme cascade is then terminated at each step by the use of apyrase to degrade unincorporated dNTPs and generated ATP, thus facilitating the subsequent rounds of nucleotide addition. In the continuous RNA-dependent polymerase assay described here, apyrase is not included, thus preventing the termination of the increasing signal. The present invention utilizes the continuous increasing signal to aid in the detection and to lower the detection limits. This difference in the enzyme cascade allows the number of nucleotides incorporated into the elongating RNA or DNA chain to be monitored continuously over time.

Definitions and General Methods

The practice of the present invention will employ, unless otherwise indicated, conventional methods of chemistry, biochemistry, molecular biology, virology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods in Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Unless stated otherwise, the following terms and phrases as used herein are intended to have the following meanings:

The terms “nucleic acid”, “polynucleotide” or “oligonucleotide” mean polymers of nucleotide monomers or analogs thereof, including double and single stranded deoxyribonucleotides, ribonucleotides, alpha-anomeric forms thereof, and the like. Usually, the monomers are linked by phosphodiester linkages, where the term “phosphodiester linkage” refers to phosphodiester bonds or bonds including phosphate analogs thereof, including associated counterions, e.g., H⁺, NH₄⁺, Na⁺.

“Nucleoside” refers to a compound consisting of a purine nucleobase, or purine-like heterocycles, or pyrimidine nucleobase, or pyrimidine-like nucleobase e.g., adenine, guanine, cytosine, uracil, thymine, deazaadenine, deazaguanosine, and the like, linked to a pentose at the 1′-position. For example, when the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine. One of skill in the art will understand that the exact numbering system will depend on the particular compounds being combined.

“Nucleotide” refers to a phosphate ester of a nucleoside, e.g., a triphosphate ester, wherein the most common site of esterification is the hydroxyl group attached to the C-5 position of the pentose. A nucleotide is composed of three moieties: a sugar, a phosphate, and a nucleobase (Blackburn, 1996). When part of a duplex, nucleotides are also referred to as “bases” or “base pairs”. The most common naturally-occurring nucleobases, adenine (A), guanine (G), uracil (U), cytosine (C), and thymine (T) bear the hydrogen-bonding functionality that effect Watson/Crick base-pairing. A ribonucleotide is a nucleotide that contains a ribose as the pentose sugar, while a deoxyribonucleotide contains a deoxyribose as the pentose sugar.

Polynucleotides typically range in size from a few monomeric units, e.g. 5-40, to several thousands and in some cases, up to about 20,000 monomeric units. Whenever an RNA polynucleotide is represented by a sequence of letters, such as “AUGCCUG,” it will be understood that the nucleotides are represented in 5′ to 3′ order from left to right and that “A” denotes adenosine, “C” denotes cytidine, “G” denotes guanosine, and “U” denotes uridine, unless otherwise noted. Whenever a DNA polynucleotide is represented by a sequence of letters, such as “ATGCCTG,” it will be understood that the nucleotides are represented in 5′ to 3′ order from left to right and that “A” denotes deoxyadenosine, “C” denotes deoxycytidine, “G” denotes deoxyguanosine, and “T” denotes deoxythymidine, unless otherwise noted.

The term “Watson/Crick base-pairing” refers to a pattern of specific pairs of nucleotides, and analogs thereof, that bind together through sequence-specific hydrogen-bonds, e.g. A pairs with T or U, and G pairs with C.

The term “nucleic acid or nucleotide analogs” refers to analogs of nucleic acids made from monomeric nucleotide analog units, and possessing some of the qualities and properties associated with nucleic acids. Nucleotide analogs may have modified (i) nucleobase moieties, e.g. C-5-propyne pyrimidine, pseudo-isocytidine and isoguanosine, (ii) sugar moieties, e.g. 2′-O-alkyl ribonucleotides, and/or (iii) internucleotide moieties, e.g. 3′-N-phosphoramidate. See Englisch, U. and Gauss, D. “Chemically modified oligonucleotides as probes and inhibitors”, Angew. Chem. Int. Ed. Engl. 30:613-29 (1991). A class of analogs where the sugar and internucleotide moieties have been replaced with an 2-aminoethylglycine amide backbone polymer is peptide nucleic acids PNA. See P. Nielsen et al., Science 254:1497-1500 (1991).

The phrase “positive stranded genome” of a virus is one in which the genome, whether RNA or DNA, is single-stranded and which encodes a viral polypeptide(s). Examples of positive stranded RNA viruses include the virus families Flaviviridae, Togaviridae, Coronaviridae, Retroviridae, Picornaviridae, and Caliciviridae. See Fields & Knipe (1986) “Fundamental Virology” (Raven Press, NY).

The term “end-point” measurements refers to a method where data collection occurs only once the reaction has been stopped.

The term “real-time continuous” refers to a method where data collection occurs through periodic monitoring during the course of the polymerization reaction.

The term “enzyme/template mixture” refers to combinations of an “enzyme mixture” comprising polymerase (RdRp or RdDp), luciferase, ATP sulfurylase and the “hybridized polynucleotide”. The “hybridized polynucleotide” refers to the primer polynucleotide after it has been hybridized to a template polynucleotide.

An “RNA-dependent polymerase” refers to an enzyme that produces a polynucleotide sequence (DNA or RNA), complementary to a pre-existing template polyribonucleotide (RNA). The RNA-dependent polymerase may be either an RNA-dependent RNA polymerase or an RNA-dependent DNA polymerase. The RNA-dependent polymerase may be either an RNA viral polymerase or replicase or RNA-dependent cellular polymerase.

A “cellular polymerase” is a polymerase derived from a cell. The cell may be prokaryotic or eukaryotic. A “mammalian RNA polymerase II” is an RNA polymerase II derived from a mammal. The polymerase is optionally naturally occurring, or artificially (e.g., recombinantly) produced. A “human RNA polymerase II” is an RNA polymerase II derived from a human. The polymerase is optionally naturally occurring, or artificially (e.g., recombinantly) produced. A “murine RNA polymerase II” is an RNA polymerase II derived from a mouse. The polymerase is optionally naturally occurring, or artificially (e.g., recombinantly) produced.

The term “substrate mixture” refers to refers to combinations of one or more ribonucleotide triphosphates for an RdRp (GTP, UTP, CTP and ATP or nucleotide analogs thereof), or deoxyribonucleotide triphosphates dATP for an RdDp (dGTP, dTTP, dCTP or deoxyribonucleotide analogs thereof); adenosine 5′-phosphosulfate (APS); and D-luciferin. When characterizing the effect of inhibitors on a polymerase, the inhibitor is usually added to the substrate mixture. Alternatively, the inhibitor may be pre-incubated with the polymerase by adding it to enzyme/template mixture prior to initiating the reaction with the substrate mixture.

The term “PPᵢ” refers to inorganic pyrophosphate. The term “PPᵢ detection mixture”, as used herein refers to combinations of enzymes such as ATP sulfurylase, luciferase and substrates such as APS and D-luciferin.

The terms “hybridize” and “hybridization” refers to the process in which complementary sequences of single stranded DNA or RNA pair up and form a double helix. The two strands are hydrogen bonded together in an anti-parallel configuration, with individual base pairs forming Watson/Crick base pairs. Hybridization conditions that allow only the hybridization of complementary sequences of identical or very similar sequences are known as “stringent conditions”. Stringent conditions include high temperature, low ionic strength and high formamide concentrations. Moderate stringent conditions will allow nucleic acids with reduced but still substantial similarity to hybridize to each other. Those of ordinary skill will readily recognize that hybridization and wash conditions can be determined to provide desired stringency.

RNA Viruses

The methods of the present invention are particularly well suited for studying and developing inhibitors of RNA viruses, including both positive-stranded and negative-stranded viruses. In a preferred embodiment, the methods of the present invention can be used to discover and characterize inhibitors of the polymerases from RNA viruses having a positive stranded genome and the various associated types.

A salient characteristic of viruses with RNA genomes is that they have relatively high rates of spontaneous mutation reportedly on the order of 10⁻³ to 10⁻⁴ per incorporated nucleotide. See Fields & Knipe (1986) “Fundamental Virology” (Raven Press, NY). Since heterogeneity and fluidity of genotype are inherent in RNA viruses, there are multiple types/subtypes, within the species that may be virulent or avirulent. For example, the propagation, identification, detection, and isolation of various HCV types or isolates is documented in the literature. The numbering of the HCV-1 genome and amino acid residue sequences is as described in Choo et al. Brit. Med. Bull., 46:423-441 (1990). The disclosure herein allows the detection of the various types.

Positive-Stranded RNA Viruses

Examples of positive-stranded virus families and specific members of the family, where the methods of present invention are useful are shown below:

Flaviviridae such as Hepatitis C virus, West Nile virus, Dengue virus, yellow fever virus, Japanese encephalitis virus, St. Louis encephalitis virus and central European encephalitis virus. See Fields Virology 3^{rd} edition, Edited by B. N. Fields, D. M. Knipe, and P. M. Howley, Lippincott Raven, Philadelphia Pa. (1996), Chapters 30-33 for Flaviviridae (pp. 931-1074), the disclosure of which is hereby incorporated by reference in its entirety.

Togaviridae such as Rubella, Western equine encephalitis virus, Venezuelan equine encephalitis virus, Eastern equine encephalitis virus, Semiliki Forest virus, and Sinbis virus. See Fields Virology 3^{rd} edition, Edited by B. N. Fields, D. M. Knipe, and P. M. Howley, Lippincott Raven, Philadelphia Pa. (1996), Chapters 27-29 for Togaviridae (pp. 825-930), the disclosure of which is hereby incorporated by reference in its entirety.

Coronaviridae, such as human respiratory coronaviruses HCV-229E & OC43 and Severe Acute Respiratory Syndrome coronavirus (SARS-CoV). See Fields Virology 3^{rd} edition, Edited by B. N. Fields, D. M. Knipe, and P. M. Howley, Lippincott Raven, Philadelphia Pa. (1996), Chapters 34-35 for Coronaviridae (pp. 1075-1104), the disclosure of which is hereby incorporated by reference in its entirety.

Retroviridae, such as HIV, and HTLV-I & II. See Fields Virology 3^{rd} edition, Edited by B. N. Fields, D. M. Knipe, and P. M. Howley, Lippincott Raven, Philadelphia Pa. (1996), Chapters 58-62 for Retroviridae (pp. 1767-1996), the disclosure of which is hereby incorporated by reference in its entirety.

Picornaviridae, such as human rhinoviruses, poliovirus, coxsackievirus A & B, hepatitis A virus, Echovirus, encephalomyocarditis virus and Theiler's virus. See Fields Virology 3^{rd} edition, Edited by B. N. Fields, D. M. Knipe, and P. M. Howley, Lippincott Raven, Philadelphia Pa. (1996), Chapters 21-24 (pp. 609-735) the disclosure of which is hereby incorporated by reference in its entirety.

Caliciviridae such as the Norwalk Group of viruses. See Fields Virology 3^{rd} edition, Edited by B. N. Fields, D. M. Knipe, and P. M. Howley, Lippincott Raven, Philadelphia Pa. (1996), Chapter 25 for Calciviridae (pp. 784-804), the disclosure of which is hereby incorporated by reference in its entirety.

Hepatitis C Virus

One illustrative example of an RNA virus in which the methods of the present invention may be used to discover and characterize inhibitors and antiviral therapies is HCV. HCV, a positive stranded RNA virus, infects nearly 3% of the world's population. Infection is associated with liver cirrhosis and, in some cases, hepatocellular carcinoma. Following HCV infection, the viral RNA is translated into a ˜3,000-residue polyprotein that is subsequently cleaved into individual components by host peptidases and virally encoded proteases. The HCV genome encodes at least 10 structural and nonstructural proteins required for viral assembly and replication, respectively.

The non-structural region of HCV starts with NS2, a hydrophobic protein of 24 kDa whose function is unknown. NS3, a protein of 68 kDa which follows NS2 in the polyprotein, is predicted to have two functional domains: a serine protease domain in the first 200 amino-terminal amino acids, and an RNA-dependent ATPase domain at the carboxy terminus. The gene region corresponding to NS4 codes for NS4A and NS4B, two hydrophobic proteins of 6 and 26 kDa, respectively, whose functions have not yet been clarified. The gene corresponding to NS5 also codes for two proteins, NS5A and NS5B, of 56 and 65 kDa, respectively.

HCV NS5B Polymerase

The RdRp activity of HCV resides in nonstructural protein 5B (NS5B), a 66 kDa membrane-associated protein encoded at the HCV polyprotein C-terminus (residues 2,420-3,010). This gene product was initially identified as an RdRp by the presence of the hallmark Gly-Asp-Asp sequence, which is involved in binding the Mg²⁺ ions essential for polymerase function. HCV NS5B is active in the absence of host or viral cofactors and in vitro is capable of replicating the entire HCV genome. It is now believed that in vivo NS5B is anchored to the endoplasmic reticulum as part of a larger viral replication complex that includes the NS3 helicase.

Nucleotidyl polymerases, such as NS5B, bind a nucleic acid template and direct the elongation of a strand complementary to the template strand by sequential addition of nucleotides. Nucleotidyl polymerases generally require a nucleic acid primer in order to bind to and initiate elongation on the template strand. Despite negligible sequence homology among polymerases, crystallographic studies have revealed conserved subdomain organization and arrangement of structural motifs among DNA-dependent DNA polymerases, DNA-dependent RNA polymerases and human immunodeficiency virus type 1 reverse transcriptase (HIV-1 RT), which is both an RNA- and a DNA-dependent DNA polymerase. Sequence homology within the structural motifs, which are spatially clustered and centrally located in the polymerase molecule, is essentially limited to the catalytic residues. Conserved residues include two aspartic acids, which bind the Mg²⁺ ions. Structures of polymerases in binary and ternary complexes with nucleic acid polymers and nucleotide substrates have pinpointed regions important for substrate binding, fidelity of nucleotide incorporation and processivity. See Doublie et al., Structure 7:R31-R35 (1999).

HCV NS5B has been shown to be an RNA-dependent RNA polymerase. As such, NS5B is believed to be responsible for genome replication of HCV. It has been demonstrated that NS5B alone can replicate the entire HCV genome via a copy-back mechanism initiated from the end of the 3′ untranslated region. See Lohmann et al. J. Virol. 71:8416-8428 (1997). Cellular localization studies revealed that NS5B is membrane associated and distributed in the perinuclear region. This localization coincides with the distribution of NS5A, suggesting that NS5A and NS5B may stay together after proteolytic cleavage at NS5A/NS5B. It has been postulated that the nonstructural proteins of HCV (NS3 to −5B) may assemble into membrane-associated replication complexes which are competent for authentic RNA genome replication. See Ferrari et al., J. Virol. 73:1649-1654 (1999); Hwang et al., J. Virol. 70:533-540 (1996); the disclosures of which are hereby incorporated by reference in their entirety.

Although the overall amino acid composition of HCV NS5B is not hydrophobic, there is a 21-amino acid residue hydrophobic tail that could potentially serve as a membrane anchor region. This hydrophobic tail is found in other genotypes of HCV as well as other members of the Flaviviridae family, such as the pestiviruses (e.g., Bovine Viral Diarrhea virus and Classic Swine Fever virus). A comparison of the C-terminal sequences of hepatitis C virus NS5B is disclosed in U.S. Pat. No. 6,228,576 to Delvecchio (1998), the disclosure of which is hereby incorporated by reference in its entirety.

Deletion of this hydrophobic tail of HCV NS5B releases the protein into the soluble portion of the cell, allowing for a greater recovery of soluble protein for screening for inhibitors of NS5B enzymatic activity. See Ferrari et al., J. Virology 73: 1649-1654 (1999), the disclosure of which is hereby incorporated by reference in its entirety. Additionally, the production of soluble protein produced has allowed for determination of the structure of the protein via x-ray crystallography. See Lesburg et al., Nat. Struct. Biol. 6:937-943 (1999), the disclosure of which is hereby incorporated by reference in its entirety. This information can be used to discover or to guide the development of inhibitors. These inhibitors of NS5B potentially could have antiviral activity and, thus, could be used as therapeutic agents for the treatment of viruses of the Flaviviridae family, particularly HCV. Other viruses for which the inhibitor may be used include flaviviruses, such as Yellow Fever virus, West Nile virus, Venezuelan Equine encephalitis virus and Dengue virus types 1-4; and pestiviruses, such as Bovine Viral Diarrhea virus and Classic Swine Fever, among others.

HCV NS5B may be obtained as described in U.S. Pat. No. 5,981,247 to Hagedorn et al., (1999), the disclosure of which is hereby incorporated by reference in its entirety. Hagedorn et al., provides a modified recombinant HCV NS5B protein having RdRp activity. The recombinant protein may be expressed in a mammalian host such as baby hamster kidney (BHK) cells or bacterial cells such as E-coli. The modification includes addition at the amino terminus of a methionine residue and optionally from 1-20 additional amino acids interposed between the N-terminal methionine and the N-terminal serine of the unmodified NS5B gene product. The modification also includes deletion at the amino terminus of up to 9 amino acids to provide an amino-terminal methionine. Other soluble HCV NS5B polypeptides that may be used, e.g., a polypeptide that deletes 21 amino acids at the C-terminus of the protein, are described in Ferrari et al., J. Virol. 73: 1649-54 (1999).

The HCV NS5B polymerase reaction in the presence of the extension primer and a template is carried out using an polymerase that will incorporate ribonucleotides. Any suitable NS5B polymerase may conveniently be used and many are known in the art and reported in the literature. The precise reaction conditions, concentrations of reactants etc. may readily be determined for each system according to choice. However, it may be advantageous to use an excess of NS5B polymerase over primer/template to ensure that all free 3′ ends are extended.

Poliovirus

Poliovirus is a useful model system for studying RNA viruses and their replication. RNA structures at the 3′ end of the poliovirus genome, and possibly at the 5′ end of the genome, specify the site of assembly of the replication complex, and both viral and host factors have been implicated in replicase assembly and/or function. After complex assembly, PV 3D^{pol} initiates RNA synthesis by using the protein primer 3B (VPg). 3D^{pol} has been intensively studied for many years because of its role in poliovirus genome replication. Therefore, this enzyme is an ideal model system to use for studying mechanisms of RdRp action and for elucidating RdRp structure-function relationships. Elucidation of these mechanisms and relationships may be useful in identifying inhibitors of RdRp enzymes. In addition, although there is an oral vaccine for polio available, the disease is still a problem in some parts of the world, particularly in India. In 2002, there were over 3000 cases of polio worldwide, with almost half of those cases in India. Thus, the identification of inhibitors of 3D^{pol} may be useful for combating the disease. Methods for isolating or recombinantly producing 3D^{pol} are well-known in the art. See, e.g., Ward et al., J. Virology 62: 558-562 (1988); Arnold et al., J. Biol. Chem., 274: 2706-2716 (1999).

Negative-Stranded RNA Viruses

There are a large number of negative-stranded RNA viruses that are relevant to human health, including members of the families of Rhabdoviridae, Paramyxoviridae and Orthomyxoviridae. See Fundamental Virology, 3^{rd} edition, edited by B. N. Fields, D. M. Knipe, and P. M. Howley, Lippincott-Raven, Philadelphia, Pa. (1996) Chapters 19-21. Rabies virus is a member of the Rhabdoviridae family while Sendai virus, Mumps virus and Measles virus are members of the Paramyxoviridae family. The family Orthomyxoviridae includes the influenza virus, which infects 10% to 20% of the American population each year as well as many millions of people worldwide. Influenza also causes many deaths, especially among the elderly and those with a compromised immune system. There are many different strains of the influenza virus at any one time, and the strains change over time. Further, some strains are highly virulent, and cause mass epidemics, such as the one in 1918 that killed over 20 million people worldwide. An influenza vaccine (flu shot) reduces the likelihood of an individual getting influenza; however, the vaccine must be received yearly because influenza strains change rapidly. Further, although there are a number of antiviral medications that can be used to treat influenza, there is still a need for better medications that can inhibit the influenza virus. Thus, a method for identifying inhibitors of the influenza RdRp would be useful in developing anti-influenza drugs.

The influenza RdRp is a complex composed of three viral proteins: PB1, PB2 and PA. A nucleoprotein (NP) that binds to the viral RNA may also be necessary for transcription. The RdRp complex may be isolated by any method known in the art or may be produced recombinantly. See, e.g., Lee et al., Nucl. Acids Res. 30: 429-38 (2002). The recombinantly produced complex can initiate transcription on an RNA ligand comprising pre-annealed 5′- and 3′-ends or on an RNA ligand formed by sequential addition of synthetic RNA oligonucleotides corresponding to the 5′-end and 3′-end of a viral RNA. See, e.g., Lee et al., supra and Lee et al., Nucl. Acids Res. 31: 1624-32 (2003). Thus, following the teachings of the specification, one having ordinary skill in the art can use the influenza RdRp in the methods of the specification to measure the activity of the influenza RdRp and to identify inhibitors thereof.

Other Polymerases

Non-Viral RNA-Dependent RNA Polymerases

Over the past several years, it has become clear that many eukaryotes encode endogenous RdRps, which are likely involved in post-transcriptional gene silencing (RNA silencing) and viral host defense. See Plasterk, Science 296:1263-65 (2002); Zamore, Science 296:1265-69 (2002); Ahlquist, Science 296:1270-73 (2002). RdRps have been identified in diverse organisms including amoebae, such as the slime mold Dictyostelium discoideum; fungi, such as Neurospora crassa and Schizosaccharomyces pombe; plants, such as tomato, Arabidopsis and Nicotiana; and worms, such as Caenorhabditis elegans. More than one RdRp have been identified in organisms including Arabidopsis, Dictyostelium and C. elegans. See Plasterk, supra. An RdRp from N. crassa has been recombinantly expressed and purified, and shown to have RNA-dependent RNA polymerase activity in vitro. See Makeyev et al., Mol. Cell 10:1417-27 (2002). Thus, following the teachings of the specification, one having ordinary skill in the art can use a non-viral RdRp in the methods of the specification to measure the activity of the non-viral RdRp and to identify inhibitors thereof.

These methods are useful due to the role of non-viral RdRps in, inter alia, post-transcriptional gene silencing and viral host defense.

Reverse Transcriptases

The reverse transcriptase (RT) of HIV-1 plays an indispensable role in the life cycle of the virus. Its premier function is the synthesis of a double-stranded DNA copy of the RNA genome for integration into the host chromosome. This is achieved by the concerted application of a number of innate activities including minus-strand DNA synthesis via an RNA-dependent DNA polymerase activity, concomitant degradation of the template RNA strand via an RNase H activity, and plus-strand DNA synthesis via a DNA-dependent DNA polymerase activity. See Baltimore, D. Nature 226:1209 (1970); Temin, H. M. and Mizutani, S. Nature 226:1211 (1970); Gilboa, E. et al. Cell 18:93-100 (1979); Peliska, J. A. and Benkovic, S. J. Science 258:1112-1118 (1992). Because the cells HIV-1 infects contain no endogenous RT, it must also possess a mechanism to ensure its packaging into the mature viral particle to guarantee its presence in the succeeding infection.

The methods of the invention are particularly advantageous when used for studying and developing inhibitors of retroviruses or other viruses otherwise replicated by an RNA intermediate. Thus, the present invention can also be used to develop inhibitors to RNA-dependent DNA polymerases, such as a reverse transcriptase found in all retroviruses. Retrovirus RNA-dependent DNA polymerases that can be targeted for inhibitor development include HIV-1, HIV-2, the human T-cell leukemia (HTLV) viruses such as HTLV-1 and HTLV-2, adult T-cell leukemia (ATL), the human immunodeficiency viruses such as HIV-1 and HIV-2, simian immunodeficiency virus (SIV), feline leukemia virus (FeLV) and feline immunodeficiency virus (FIV). Reverse transcriptase from retroviruses may be obtained commercially, purified or recombinantly produced using methods known in the art. For example, HIV RT may be purchased from, e.g., Amersham Biosciences, or may be recombinantly produced. See, e.g., Kim et al., J. Biol. Chem. 274:27666-27673 (1999).

Primer Polynucleotides

The first steps of the disclosed methods comprise providing a primer oligonucleotide having a 3′OH, which is allowed to hybridize to a template RNA. For this purpose, a wide variety of RNAs may be employed as a primer, including synthetic RNA oligomers or derivatives thereof, cellular RNA, amplified RNA or purified RNA species such as rRNA, tRNA or preferably, mRNA, etc.

In one embodiment, the primer comprises a sequence of sufficient length and appropriate sequence to hybridize with the template RNA at or near the 3′ end of the template RNA. Depending upon the application, this hybridizing sequence may be random, specific or a combination of random and specific sequences. For example, a primer population comprising random hybridizing sequences provides a “universal” primer set capable of targeting any RNA species. In a preferred embodiment, a 3′-OH-containing primer comprising poly(G) may be used to hybridize to a template comprising a synthetic poly(C) polynucleotide. In one embodiment, the primer may be on the same strand as the template RNA, wherein hybridization would occur in a fold back mechanism.

In other embodiments, a 3′-OH-containing primers comprising poly(U) sequences may be used to hybridize to poly(A) tails of mRNA; primers comprising predetermined specific sequences may be used to target particular, predetermined RNA species comprising complementary sequences. Primers comprising a random region joined to a wobble nucleotide (A, C or G) joined to a poly(U) region may be used to target mRNA poly(A) junctions. In a preferred embodiment, primers are RNA oligonucleotides complementary to and deduced from known HCV or other viral sequences from the Flaviviridae, Togaviridae, Coronaviridae, Retroviridae, Picornaviridae, and Caliciviridae families of viruses known in the art. For example, the sequence of HCV was described in U.S. Pat. No. 5,714,596 to Houghten et al., (1998) and U.S. Pat. No. 6,074,816 to Houghten et al., (2000), the disclosures of which are hereby incorporated by reference in their entirety. Many other RNA viral sequences are known in the art. In cases in which the RNA sequence is presented as a cDNA, one of skill in the art would appreciate that RNA nucleotides should be used rather that deoxynucleotides.

In a preferred embodiment, the primer polynucleotide is an oligonucleotide. In the context of this invention, the term “oligonucleotide” refers to an oligomer of ribonucleic acid or deoxyribonucleic acid. This term includes oligomers consisting of naturally occurring bases, sugars and intersugar (backbone) linkages as well as oligomers having non-naturally occurring portions that function similarly. Such modified or substituted oligonucleotides are often preferred over native forms because of properties such as, for example, decrease background light induced in the luciferase reaction and increased stability in the presence of nucleases.

All such oligonucleotides are comprehended by this invention so long as they function effectively to hybridize with the template. The oligonucleotides in accordance with this invention preferably comprises from about 5 to about 50 nucleic acid base units. It is more preferred that such oligonucleotides comprise from about 8 to about 30 nucleic acid base units, and still more preferred to have from about 12 to about 25 nucleic acid base units wherein a nucleic acid base unit is a base-sugar combination suitably bound to adjacent nucleic acid base unit through phosphodiester or other bonds.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed as the actual synthesis of the oligonucleotides is routine and within the skill of the art. It is also well known to use similar techniques to prepare other oligonucleotides such as the phosphorothioates and alkylated derivatives.

Suitable reaction conditions for effecting hybridization between the target RNA and first primer are known in the art, readily ascertained empirically, or have been described or exemplified herein.

Template Polynucleotides

As used herein, the term “template directed” elongation refers to an RNA-dependent polymerase activity that synthesizes an RNA or DNA that is complementary to the template sequence. In contrast, “terminal transferase activity” as used herein refers to the non-templated addition of nucleotides to the 3′ end of an RNA or DNA strand. A single enzyme may possess both activities.

The template comprises an RNA polynucleotide of sequence and length sufficient to hybridize with the primer RNA polynucleotide and then bind the viral polymerase such as HCV NS5B and allow for primer-dependent template-directed elongation to proceed. In one embodiment, the elongation proceeds for at least about 1 base pair, preferably between about 2 and about 5 base pairs, preferably about 10 base pairs, preferably between about 10 and about 99 base pairs, preferably about 100 base pairs, preferably between about 100 and about 999 base pairs, more preferably about 1,000 base pairs, preferably between about 1000 and about 9999 base pairs, even more preferably about 10,000 base pairs. Alternatively, in some applications the template may be an RNA sequence of sequence and length sufficient to allow primer dependent elongation reactions to proceed for about 100,000 base pairs, or about 1,000,000 base pairs or more. Depending on the application, the template sequence may be limited to single nucleotides such as poly(G), poly(C), or poly(A), or combinations of two, or three nucleotides or derivatives thereof arranged in random, specific or a combination of random and specific sequences. Generally, poly(U) or oligomers containing U should be avoided because ATP is unsuitable as a substrate in this coupled assay, although certain types of ATP analogs may be used.

In a preferred embodiment, an 3′-OH-containing primer comprising poly(C) may hybridize to a template polynucleotide comprising synthetic poly(G). In another embodiment, an 3′-OH-containing primer comprising poly(G) may be used to hybridize to a template polynucleotide comprising synthetic poly(C). In other embodiments, 3′-OH-containing primers comprising poly(U) may be used to hybridize to template mRNAs by hybridizing to poly(A) tails. In a preferred embodiment, HCV, or other RNA virus such as West Nile virus, Dengue virus, yellow fever virus or Venezuelan equine encephalitis virus, or other viral sequences from the Flaviviridae, Togaviridae, Coronaviridae, Retroviridae, Picornaviridae, and Caliciviridae families of viruses are used as templates. In another embodiment, a template polynucleotide from a negative stand RNA virus or from a non-viral RNA-dependent polymerase may be used. These sequences may be of known or unknown sequence.

A potential problem that has previously been observed with PPᵢ-based sequencing methods is that ATP, when used in the polymerization reaction, interferes in the subsequent luciferase-based detection reaction by acting as a substrate for the luciferase enzyme. This may be reduced or avoided by using, in place of adenosine triphosphate (ATP), an ATP analogue which is capable of acting as a substrate for a polymerase but incapable of acting as a substrate for a said PPᵢ-detection enzyme. One such analogue is ATPαS. Alternatively, templates devoid of U will avoid use of ATP as substrate.

A “target” refers to a polynucleotide comprising a sequence for hybridization by a primer or probe oligonucleotide.

In accordance with this invention, a person of ordinary skill in the art will understand that mRNA includes not only the sequence information to encode a protein using the three letter genetic code, but also associated ribonucleotides which form regions known as the 5′-untranslated region, the 3′-untranslated region, and the 5′ cap region. These ribonucleotides also may form various secondary structures. Thus, for example, when the template is HCV genomic RNA, primer oligonucleotides may be formulated in accordance with this invention that are targeted wholly or in part to these associated ribonucleotides as well as to the coding ribonucleotides.

In a preferred embodiment, the template RNA is purified. In some applications, synthetic RNAs may be used as template without additional purification. Where purification of a synthetic or isolated RNA is necessary, techniques of purifying RNA are well known in the art. See for example, P. Chomczynski and N. Sacchi, Anal. Biochem. 162:156-159 (1987), the disclosures of which are hereby incorporated by reference in their entirety. In addition, the sequence of HCV was described in U.S. Pat. No. 5,714,596 to Houghten et al., (1998) and U.S. Pat. No. 6,074,816 to Houghten et al., (2000), the disclosures of which are hereby incorporated by reference in their entirety. Many other RNA viral sequences are known in the art. In cases in which an RNA sequence is presented as a cDNA, one of skill in the art would appreciate that ribonucleotides should be used rather that deoxyribonucleotides.

The RNA template may conveniently be single-stranded, double stranded, folded and may either by immobilized on a solid support or in solution.

In a certain embodiment of this invention, the template and primer reside on a single strand of RNA, wherein the single strand has the capacity to fold back on itself forming an internal hybridized polynucleotide. In this embodiment, the single strand of RNA having the internally hybridized polynucleotide thus provides both a primer 3′OH for initiation of polymerization and a template strand for directing the identity of added bases.

Pyrophosphate (PPᵢ) Detection

Pyrophosphate (PPᵢ) can be detected by many different methods, and a number of enzymatic methods have been described in the literature. See Reeves et al., Anal. Biochem., 28, 282-287 (1969); Guillory et al., Anal. Biochem., 39, 170-180 (1971); Johnson et al.,, Anal. Biochem., 15, 273 (1968); Cook et al., Anal. Biochem. 91, 557-565 (1978); and Drake et al., Anal. Biochem. 94, 117-120 (1979).

In the present invention, it is preferred to use luciferase and luciferin in combination to identify the release of pyrophosphate since the amount of light generated is substantially proportional to the amount of pyrophosphate released. The amount of pyrophosphate released is, in turn, directly proportional to the amount of base incorporated. The amount of light can readily be estimated by a suitable light sensitive device such as a luminometer.

Luciferin-luciferase reactions to detect the release of PPᵢ are well known in the art. In particular, a method for continuous monitoring of PPᵢ release based on the enzymes ATP sulphurylase and luciferase has been developed by Nyren et al., Anal. Biochem., 151, 504-509, (1985) and termed ELIDA (Enzymatic Luminometric Inorganic Pyrophosphate Detection Assay). The use of the ELIDA method to detect PPᵢ is preferred. The method may however be modified, for example by the use of a more thermostable luciferase (White et al., Biochem. J. 319: 343-350 (1996); Hirokawa et al., Biochim. Biophys. Acta 1597: 271-79 (2002); Kajiyama et al., Biosci. Biotech. Biochem., 58: 1170-1171 (1994); Kajiyama et al., Biochemistry 32: 13795-9 (1993)) and/or ATP sulfurylase (Onda et al., Bioscience, Biotechnology and Biochemistry, 60: 1740-1742 (1996)).

This method is based on the following reactions:

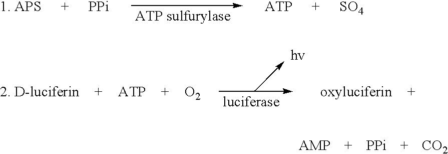

The preferred detection enzymes involved in the PPᵢ detection reaction are thus ATP sulphurylase and luciferase and are included in the detection mixture.

Due to the simplicity and rapidity of the assay techniques, it is easy to automate by using a robot apparatus where a large number of samples may be rapidly analyzed. Further, since the preferred detection and quantification is based on a luminometric reaction, this can be easily followed photometrically. The use of luminometers is well known in the art and described in the literature.

Methods of the Invention

The invention provides a coupled sequencing/detection system that is based on the following reactions:

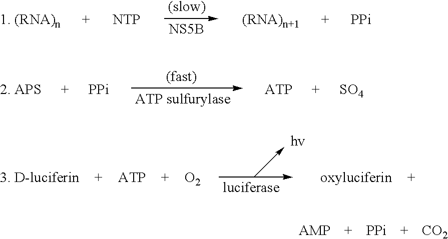

NS5B in Reaction 1 can be any RdRp. If an RNA-dependent DNA polymerase, such as HIV RT, is used, then Reaction 1 may be described by the following formula:

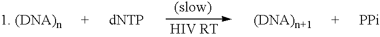

Reactions 2 and 3 are the same as those described above.

The methods of the invention are used to measure RNA-dependent polymerase activity and can be used to characterize inhibitors of polymerases from a variety of sources, including RNA viruses, such as the RdRp from HCV and RT from HIV. The method of the invention involves continuous monitoring of base incorporation reactions monitored in real time, in the presence and absence of an inhibitor. Continuous real time measurements are achieved by performing the chain extension and detection (signal generation) reactions substantially simultaneously by including the “detection enzymes” in the chain extension reaction mixture. This represents a departure from the approach reported in the PPᵢ-based sequencing procedures discussed above, in which the chain extension reaction is first performed separately as a first reaction step, followed by a separate “detection” reaction, in which the products of the extension reaction are subsequently subjected to the luciferin-luciferase based signal generation (“detection”) reactions.

To carry out this preferred embodiment of the method of the invention, a primer oligonucleotide is preincubated with a template polynucleotide and hybridization is allowed to occur, thus forming a hybridized polynucleotide. As used herein, a “hybridized polynucleotide” refers to the molecular complex that is formed when a primer polynucleotide, preferably a primer oligonucleotide, is hybridized with a template polynucleotide. The sequences of the primer oligonucleotide and the template polynucleotide are designed such that the 5′ end of the template polynucleotide will extend beyond the 3′ end of the primer polynucleotide when they are hybridized to each other. This will provide a template for the RNA-dependent polymerase to use. Generally, the template polynucleotide will be longer than the primer oligonucleotide if two separate polynucleotides are used.

In one preferred embodiment, the hybridized polynucleotide is combined with a target sample containing an RdDp. At or around the same time (i.e., soon thereafter or soon before), the detection mixture is combined with the target sample and the hybridized polynucleotide to generate a reaction mixture for the polymerase reaction to occur.

In one embodiment, the PPᵢ-detection mixture contains enzymes such as ATP sulfurylase and luciferase, which are combined in a detection mixture with APS and luciferin. In another embodiment, the PPᵢ-detection mixture contains enzymes such as ATP sulfurylase and luciferase, which are combined in a detection mixture without APS and luciferin. The APS and luciferin may be added, separately or together, to the reaction mixture before or after enzyme addition. This invention envisions that the order of addition of the enzymes and substrate may be varied to suit individual application.

In a preferred embodiment of the invention, the reaction is initiated by adding an initiation mixture to the reaction mixture. In one embodiment, the initiation mixture comprises one or more NTPs or dNTPs, depending upon whether the RNA-dependent polymerase is an RdRp or an RdDp. In a preferred embodiment, the initiation mixture comprises one or more NTPs, APS and D-luciferin. The initiation mixture may also be called the substrate mixture.

There is considerable flexibility in when each of the components of the final reaction mixture are added to the reaction mixture. For example, the primer and template may be pre-incubated to allow hybridization to occur, then followed by addition of the polymerase, other enzymes, followed by the addition of the substrate compounds such one or more NTPs or dNTPs, APS, and D-luciferin. Alternatively, the substrate compounds may be added initially to the hybridized polynucleotide or to the either of the individual nucleic acid polymers, the primer or the template, prior to allowing the hybridization to occur, followed by the addition of the enzyme components to initiate the reaction.

Initiation of the reactions used in the methods of the present invention can be accomplished by finally adding the missing components. If the substrate compounds are the missing elements, then they can be used to initiate the reactions used in the methods of the invention. Alternatively, the reactions used in the methods of the invention may be initiated by the addition of the enzyme mixture or the hybridized polynucleotide if these are the missing components of the final reaction mixture.

The term “reaction mixture” refers to the combination of all of the biochemical and chemical compounds necessary for the polymerase and PPᵢ reactions described above to occur. One of skill in the art will understand that many variations exist in the order of addition or combination of the various biochemical and chemical compounds and the invention is not limited to one particular scheme of addition or combination.

In another embodiment, when an inhibitor is part of the reaction mixture, additional variations in the order of addition are possible. The inhibitor can be preincubated with the polymerase or it can be added with the substrate compounds (one or more NTPs or dNTPs), or with the hybridized polynucleotide, or with any of the individual components or combinations of the individual components of the final reaction.

In another embodiment, where PPᵢ detection reaction is desired, the reaction mix for the polymerase reaction may include at least one or more NTPs or dNTPs, polymerase, luciferin, APS, ATP sulphurylase and luciferase. The polymerase reaction may be initiated by addition of the polymerase. More preferably, the reaction may be initiated by the addition of a nucleotide or a nucleotide analog thereof in the initiation mixture. Preferably, the detection enzymes are already present at the time the reaction is initiated. Alternatively, they may be added with the reagent that initiates the reaction.

One of skill in the art will understand that when measuring RdRp enzyme activity, parallel reactions that include and exclude the RNA substrate or the RdRp should be performed. Further, one skilled in the art will understand that when evaluating the effect of enzyme inhibitors on RdRp enzyme activity, parallel reactions that include and exclude inhibitor should be run at multiple enzyme, substrate and inhibitor concentrations. See K. M. Plowman, Enzyme Kinetics, McGraw-Hill, New York, “Inhibitor Studies,” pages 56-74.

The present invention permits PPᵢ release to be detected during the polymerase reaction giving a real-time signal. The RdRp reactions may be continuously monitored in real-time. A procedure for rapid detection of PPᵢ release from an RdRp reaction is enabled by the present invention. The ELIDA reactions have been estimated to take place in less than 2 seconds. See Nyren et al., Anal. Biochem. 151:504-509 (1985). The rate limiting step is the conversion of PPᵢ to ATP by ATP sulphurylase, while the luciferase reaction is fast and has been estimated to take less than 0.2 seconds. It will be seen therefore that very fast reaction times are possible, enabling real-time detection. The reaction times could further be decreased by using a more thermostable luciferase or simply more luciferase.

The method of the present invention does not employ the enzyme apyrase in the PPᵢ detection enzyme cascade. Apyrase is used in PYROSEQUENCING™ to hydrolyze excess NTPs, dNTPs, and ATP, limiting potential substrates for luciferase and restoring the light signal baseline. See Nyren et al., Anal. Biochem. 151:504-9 (1985); Nyren, Anal. Biochem. 167:235-238 (1987); Ronaghi et al., Anal. Biochem. 242:84-89 (1996). The significance of not using apyrase in the enzyme cascade is that the light signal builds or intensifies as the reaction proceeds. Thus, the present invention is able to utilize the increasing signal to aid in detecting RNA-dependent polymerase activity or to aid in characterizing inhibitors of RNA-dependent polymerase activity. For example, an intensifying signal allows continuous monitoring and greater flexibility in choosing reaction component concentrations, such as polymerase concentration. Thus, the method of the present invention may be performed in a single reaction step involving a multi-enzyme reaction mixture, e.g., a three-enzyme mixture.

Light Detectors

The present invention requires a luminometer for detecting the light generated by the luciferase reaction. One suitable luminometer is sold by Molecular Devices Corporation, 1311 Orleans Drive, Sunnyvale Calif., 94089 USA. The Lmax™ luminometer is a microplate based instrument designed for detecting chemiluminesence and bioluminesence. The system is well suited as a detector for luciferase based reactions. Other luminometers are known to those having skill in the art and may be used.

In carrying out the method of the invention, any possible contamination of the reagents e.g. the NTP solutions, by PPᵢ is undesirable. Indeed, it is desirable to avoid contamination of any sort and the use of high purity or carefully purified reagents is preferred, e.g. to avoid contamination by kinases, phosphatases or nucleases.

Reaction efficiency requires the inclusion of divalent cations such as Mg²⁺ ions in the NTP and/or polymerase reagent solutions.

### EXAMPLES

The following descriptions of particular embodiments and examples are offered by way of illustration and not by way of limitation.

### Example 1

### HCV Polymerase

Materials

Chemical reagents for assay-related buffers were obtained from Sigma (St. Louis, Mo.), USB (Cleveland, Ohio), GibcoBRL (Rockville, Md.), and Boehringer Mannheim/Roche (Indianapolis, Ind.). Purified firefly luciferase and D-luciferin were obtained from Promega (Madison, Wis.). Adenosine 5′-triphosphate (ATP) sulfurylase and adenosine 5′-phosphosulfate (APS) were obtained from Sigma (St. Louis, Mo.). Poly(C) homopolymer was from Amersham, and biotin-oligo(G)₁₂ was from Oligos, Etc. (Wilsonville, Oreg.). Microliter 1 polystyrene immunoassay plates for the polymerase/luciferase assay were from Dynex (Chantilly, Va.). An Lmax™ microtiter plate luminometer from Molecular Devices Corporation (Sunnyvale, Calif.) was used. The HCV NS5BΔ55 enzyme was prepared as described by Ferrari et al., J. Virol. 73:1649-1654 (1999).

Polymerase Assays

Polymerase assays were performed at room temperature using a 100 μl reaction mix in a 96-well plate. Final reaction conditions for NS5B were modified from those used by Ferrari et al. J. Virol. 73:1649-54 (1999) [20 mM Hepes pH 7.3; 7.5 mM DTT; 20 U/ml RNAsin; 0.5 μg/ml oligo(G)₁₂; 5 μg/ml poly(C); 1 μM GTP; 10 mM MgCl₂; 60 mM NaCl; 100 μg/ml BSA; and NS5B]. NS5B assay mixtures were supplemented with components for the ATP sulfurylase and luciferase coupled enzyme reactions [0.5 mM coenzyme A (CoA); 310 μM D-luciferin; 1 nM luciferase; 5 μM APS; 0.03-0.3 U ATP sulfurylase (see Brief Description of the Drawings for specific conditions)]. Typically, the oligo(G)₁₂ and poly(C) components (or heteropolymer primer and template) were preincubated at room temperature for 15 minutes prior to the addition of NS5B polymerase, luciferase, and ATP sulfurylase (the enzyme/template mix).

To test the effect of inhibitors on the NS5B polymerase reaction, compounds were diluted in the appropriate diluents and added to the GTP-containing mixture. Microtiter plates containing the reaction samples were immediately transferred to the luminometer for detection of the light signal generated over time, taking a 0.2-1.0 s reading of each well every 30-60 s (time intervals depend on the number of samples tested). Readings were monitored in comparison to a no-NS5B reaction as a control for ATP generation in the absence of polymerase activity. Initial velocities were determined from non-linear regression assuming pseudo-first order kinetics [P=S₀(1−e^{-kt})] and k_{cat} and Kₘ subsequently calculated by fitting estimated initial velocities to the Michaelis-Menten equation [Prism 3.0 (GraphPad Software, San Diego, Calif.)]. Data collection was restricted to PPᵢ concentrations <100 nM to avoid the influence of substrate depletion and/or product inhibition.

To test the effect of NTPs on the ATP sulfurylase/luciferase coupled read-out system, control reactions for NS5B assays (with 0.3 U ATP sulfurylase) were assembled as above omitting RdRp and RNA. Reactions were initiated by addition of nucleotide (1 μM final). To reduce free PPᵢ in commercial NTPs, 10 mM stocks were pretreated with inorganic pyrophosphatase. A 10 μl aliquot of the NTP stock was incubated with 0.5 U of thermostable inorganic pyrophosphatase for 10 minutes at 75° C.; untreated NTP samples were processed in parallel as controls. Treated and untreated NTPs were diluted to 100 μM working stocks for subsequent assays.

The effect of preincubating the polymerase with RNA template was assessed by combining (at various time points) NS5B, poly(C), and oligo(G)₁₂ at twice the final reaction concentration and initiated by addition of an equal volume of a 2× reaction mix containing luciferase, ATP sulfurylase, CoA, GTP, APS, and D-luciferin. Data were typically collected for 30 minutes.

The assay was assembled based on the reaction conditions described previously [Ferrari et al., J. Virol. 73:1649-1654 (1999)] with the incorporation of reagents used for the light-based detection of free pyrophosphate [Nyren et al., Anal. Biochem. 151:504-509 (1985)]. The light generation system is used in place of radiolabeled nucleotide and the subsequent detection of its nucleotide incorporation into elongated products. As in the PYROSEQUENCING™ enzyme cascade, the amount of light generated is proportional to the levels of pyrophosphate generated in the reaction, which are produced as by-products of the NS5B-catalyzed polymerization reaction. An unexpected benefit of modifying the PYROSEQUENCING™ enzyme cascade to exclude apyrase is that the light signal increases proportionally to the number of nucleotides incorporated and is maintained at a level reflecting incorporation, due to the recycling of pyrophosphate released in the luciferase reaction, and the generation of new pyrophosphate by the NS5B elongation reaction. All reactions were tested in parallel with control reactions without NS5B, which were subtracted from experimental data.

Linearity of Reaction in Relation to Concentration of NS5B

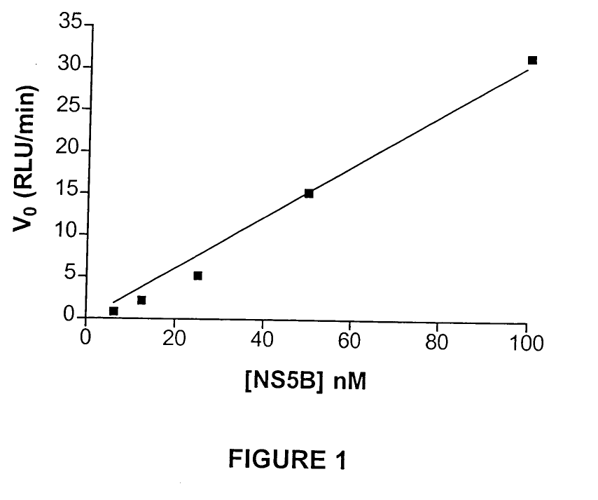

NS5B polymerase was serially diluted in enzyme storage buffer [25 mM Hepes (pH 7.5); 5 mM DTT, 0.6 M NaCl; 15% glycerol; 0.1% octylglucoside; 2 mg/ml leupeptin; 100 μM PMSF] and assayed in the presence of 1 μM GTP and the oligo (G)₁₂/poly(C) template in a 96-well plate with a final reaction volume of 100 μl at room temperature. Light was captured for 0.2 s every 60 s during a 30 minute reaction. Initial velocity values were derived from progress curve analyses and fitted by linear regression. The results are shown in FIG. 1.

A linear relationship was observed between signal detection and NS5B enzyme concentration (FIG. 1). Under these conditions in a 100 μl assay, there was a detection limit of about 6 nM NS5B, with measurable light production by 1 minute after initiation of the reaction. Sub-detectable signal from small concentrations are also possible.

### Example 2

### Dependence of Coupled Reaction on Primer and Template

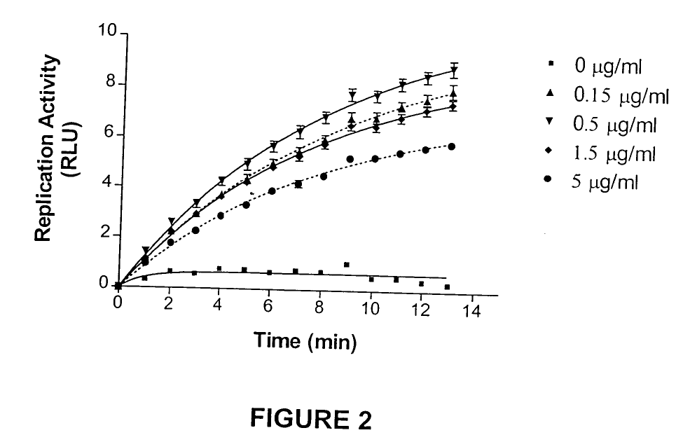

To test requirements for the oligo(G) primer in this polymerase assay, the primer was titrated over a range of concentrations to assess primer-dependency. Reactions were initiated using a fixed concentration of NS5B (20 nM) and GTP substrate (1 μM), while varying the amount of oligo(G)₁₂ primer used to anneal to the poly(C) template (5 μg/ml). Primer concentrations used in the oligo(G)₁₂/poly(C) reaction were 0 μg/ml, 0.15 μg/ml, 0.5 μg/ml, 1.5 μg/ml, and 5 μg/ml (see also FIG. 2 description). Light was captured for 0.1 s every 60 s during a 30 minute reaction. Signals from control reactions without NS5B were subtracted from experimental data.

As shown in FIG. 2, the highest level of polymerase activity was achieved with the 0.5 μg/ml oligo(G) reaction, which corresponds to the concentration used in the scintillation-proximity assay. See Ferrari et al., J. Virol. 73:1649-1654 (1999). Increasing the amount of primer used in the reaction effectively slowed the rate of polymerase activity. Low levels of light were detected in the reaction in the absence of primer. It is possible that this background signal is related to an observation that APS can non-enzymatically react with luciferin, forming a compound that activates luciferase and produces light. See Nyren et al., Anal. Biochem. 151:504-509 (1985). If so, then the generation of this background signal is apparently slow and negligible under these assay conditions.

### Example 3

### Characterizing Inhibition of HCV NS5B

The continuous non-radioactive assay of the present invention is useful in characterizing inhibitors of polymerases. To test this continuous assay for the evaluation of polymerase inhibitors, a commercially available nucleoside triphosphate analog, 3′-deoxyguanosine-5′ triphosphate (3′-dGTP; TriLink BioTechnologies, San Diego, Calif.), was used in combination with HCV NS5B.

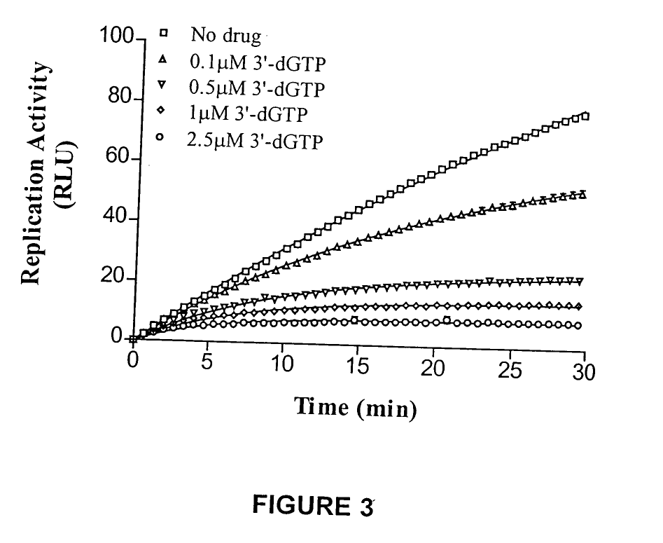

For screening the effect of inhibitors, test molecules were titrated and added to the substrate mixture (GTP, APS, and D-luciferin) prior to the addition of the enzyme/template mixture to initiate the reaction. Dose- and time-dependent inhibition of NS5B activity was observed (FIG. 3), as shown by the reduced levels of light generated during the reactions. FIG. 3 shows that increasing the concentration of inhibitor results in lower levels of light being generated. The progress curves obtained are consistent with chain termination: once elongation is inhibited by 3′-deoxyguanosine-5′ triphosphate incorporation, light production reaches a plateau. This is presumably due to recycling of the available pyrophosphate generated by NS5B-catalyzed polymerization prior to complete chain termination.

### Example 4

### 3D^{pol} Assays

Experiments were performed essentially as described in Examples 1 and 2 with the following changes: Enzyme stocks of the PV 3D^{pol} were prepared and stored in 50 mM Hepes (pH 7.3), 0.5 M NaCl, 5 mM DTT, 20% glycerol, 2 mg/L leupeptin, and 100 μM PMSF); final reaction conditions were 50 mM Hepes (pH 8.0), 2.5 mM MgCl₂, 20 U/ml RNAsin, 0.5 μg/ml oligo G₁₂, 5 μg/ml poly C, 1 μM GTP, 4 mM DTT, and 3D^{pol}; the 3D^{pol} enzyme was co-incubated along with the RNA before the reaction, and reactions were initiated by the addition of GTP, APS, and D-luciferin (the substrate mix).

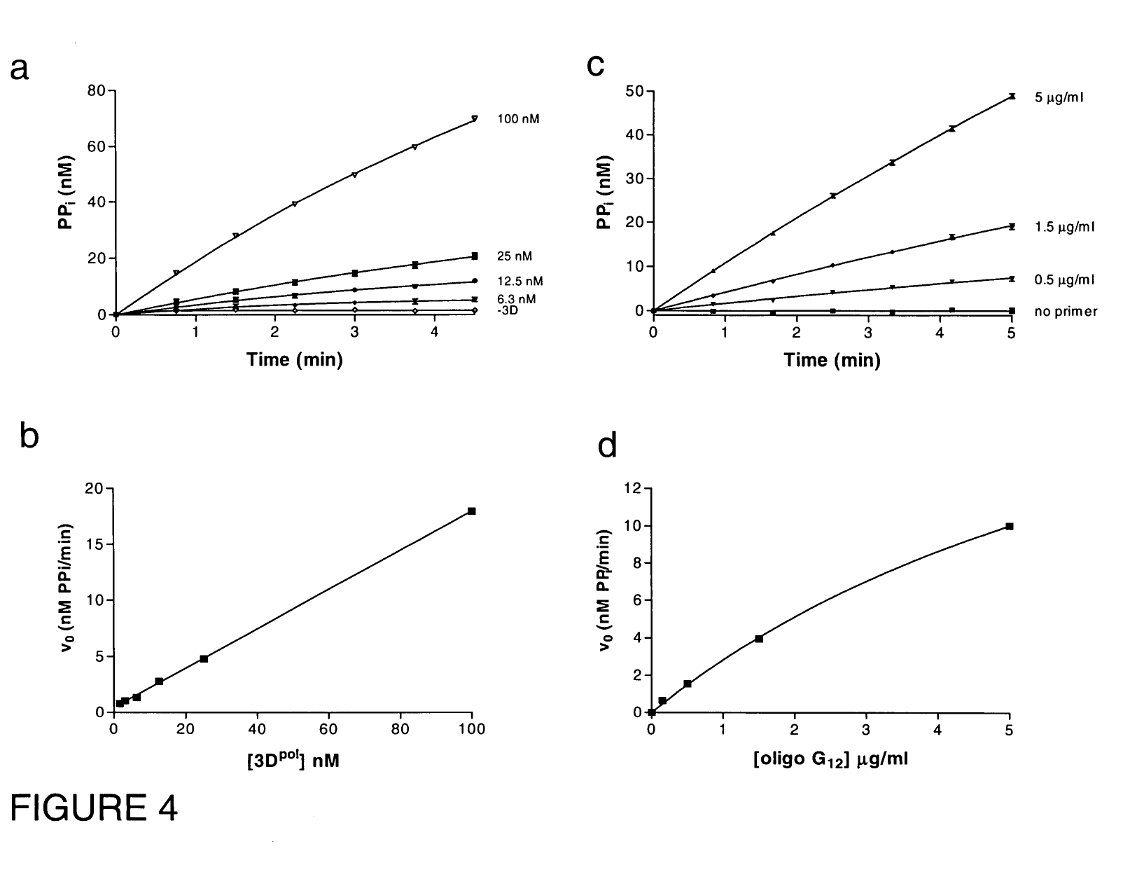

Initial experiments used the poly(C)/oligo(G)₁₂ system with PV 3D^{pol}. The reaction rate was linear with respect to enzyme concentration up to the maximum tested (100 nM) (FIGS. 4a and 4b). A measurable signal could be obtained with 6 nM 3D^{pol} in as little as 1 minute. For the subsequent studies, reactions used 10 nM 3D^{pol}. A low level of PPᵢ could be detected from reactions lacking primer (FIG. 4c). Polymerase activity increased with primer concentration, with indications of saturation at the maximum tested (5 μg/ml) (FIG. 4d).

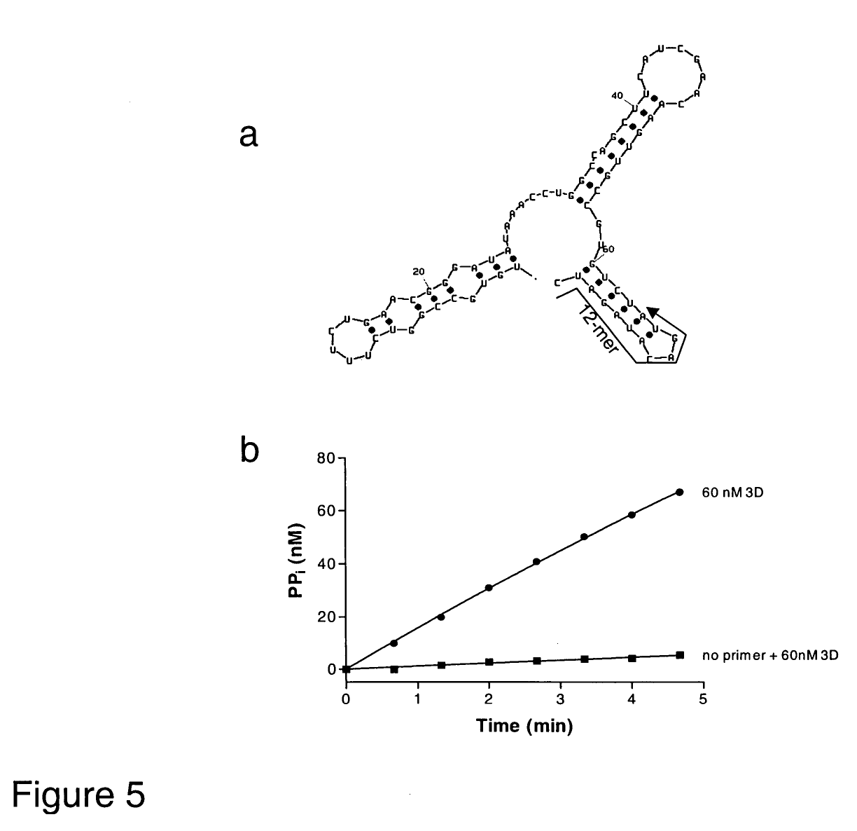

To develop an assay using non-homopolymer templates, the ability of 3D^{pol} to utilize a natural heteropolymer template and primer was tested. A 75-mer RNA oligonucleotide (FIG. 5a) was synthesized based on a sequence previously used as a template for NS5B-catalyzed reactions [D-RNA; Behrens et al., EMBO J. 15:12-22 (1996)]. Due to the strict primer-dependence of 3D^{pol}, a primer complementary to the 3′ twelve bases of D-RNA was also generated. Assays utilizing the heteropolymer template contained 100 nM of both template and primer (100 nM duplex), and 60 nM 3D^{pol}. Higher concentrations of 3D^{pol} were used in these reactions for improved signal to noise ratio. In contrast to the homopolymer assay, reactions using the heteropolymer RNA template included GTP, CTP, UTP and ATPαS, a nucleotide that is a poor substrate for luciferase. As shown in FIG. 5b, the 3D^{pol} reaction produces a strong signal compared with the no-primer control reaction (signal to noise of 10:1). Nevertheless, low levels of light can be detected from the reaction in the absence of primer (FIG. 5b). There are three possible explanations for this time-dependent signal: 1) ATPαS may non-enzymatically react with D-luciferin, forming a compound that activates luciferase and produces light, 2) 3D^{pol} may be capable of inefficient de novo (primer-independent) synthesis, as described for NS5B, or 3) some amount of self-priming may take place with this particular template (FIG. 5a). Thus, background signals need to be assessed for each RdRp and substrate, using appropriate control reactions.

Another possible source of background is direct utilization of the NTPs by the ATP sulfurylase/luciferase cascade. To address this possibility, reactions without either RdRp or template were examined. These experiments demonstrated that NTPs can act as substrates for the enzyme cascade with varying efficiencies. The Km for ATP, the normal substrate for the luciferase reaction, is 0.65±0.04 μM (under these experimental conditions, data not shown). GTP, the substrate used in the homopolymer studies reported above, had the lowest level of signal. ATPαS was also found to be a poor substrate for luciferase. Inosine triphosphate (ITP), however, was a relatively good luciferase substrate, about 10-fold better than GTP.

To eliminate contaminating PPᵢ as a source of the signal, NTP stocks were treated with inorganic pyrophosphatase. No significant changes in the initial velocity estimates were observed when NTPs were pre-treated with pyrophosphatase. Taken together, these data suggest that, under these conditions, background signals are produced as a result of utilization of NTPs by luciferase, which necessitates the use of appropriate control reactions.

### Example 5

### Reverse Transcriptase

Polymerase assays are performed essentially as described in Examples 1 and 2 with the following changes:

RT polymerase assays are performed at 37° C. using a 100 μl reaction mix in a 96-well plate. Final buffer conditions for the RT assays are 50 mM Hepes (pH 8.0), 2.5 mM MgCl₂, 20 U/ml RNAsin, 0.5 μg/ml oligo G₁₂, 5 μg/ml poly C, 1 μM dGTP, 4 mM DTT and RT. The RT enzyme is co-incubated along with the RNA before the reactions are initiated. The RT assay mixtures are supplemented and the reactions initiated by the addition of dGTP, APS, D-luciferin and a thermostable luciferase (the substrate mix). Thermostable luciferases that may be used include those disclosed in White et al., Biochem. J. 319: 343-350 (1996); Hirokawa et al., Biochim. Biophys. Acta 1597: 271-79 (2002); Kajiyama et al., Biosci. Biotech. Biochem., 58: 1170-1171 (1994); and Kajiyama et al., Biochemistry 32: 13795-9 (1993). No significant time-dependent increase in light signal was observed using firefly luciferase and RT at room temperature, 27° C., 30° C., 32° C. and 37° C. Without wishing to be bound by any theory, it is believed that RT has low polymerase activity at room temperature and that firefly luciferase is inactivated at higher temperatures. Thus, the RT assay should be performed at 37° C. using thermostable luciferase.

To test the effect of inhibitors on the RT reaction, compounds are diluted in the appropriate diluents and added to the nucleotide-containing mixture using the same reaction conditions and thermostable luciferase as described above. Microtiter plates containing the reaction samples are immediately transferred to the luminometer for detection of the light signal generated over time, taking a 0.2-1.0 s reading of each well every 30-60 s (time intervals depend on the number of samples tested). Readings are monitored in comparison to a no-RT reaction as a control for ATP generation in the absence of polymerase activity. Initial velocities are determined from non-linear regression assuming pseudo-first order kinetics [P=S₀(1−e^{-kt})] and k_{cat} and Kₘ subsequently calculated by fitting estimated initial velocities to the Michaelis-Menten equation [Prism 3.0 (GraphPad Software, San Diego, Calif.)]. Data collection is restricted to PPᵢ concentrations <100 nM to avoid the influence of substrate depletion and/or product inhibition.

All references cited herein are incorporated herein by reference to the same extent as if each individual publication or patent application was specifically and individually indicated to be incorporated herein by reference.

We claim:

1. A method for detecting RNA-dependent polymerase activity comprising:
(a) providing a primer oligonucleotide having a 3′ OH;
(b) contacting said primer oligonucleotide with a template polynucleotide and allowing hybridization to occur to form a hybridized polynucleotide;
(c) adding an RNA-dependent polymerase to said hybridized polynucleotide to produce a mixture;
(d) adding a PPᵢ detection mixture to said mixture;
(e) adding a substrate mixture comprising a nucleotide triphosphate or an analog thereof to said mixture; and
(f) measuring a product of the PPᵢ detection mixture;
wherein
apyrase is not part of the mixture and
steps (c), (d) and (e) may be performed simultaneously or separately in any order.

2. The method of claim 1, wherein said RNA-dependent polymerase is a viral RNA-dependent RNA polymerase (RdRp) selected from the group consisting of Hepatitis C virus, poliovirus, West Nile virus, Dengue virus, Human T Cell Leukemia virus, St. Louis Encephalitis virus, Yellow Fever virus and Measles virus.

3. The method of claim 2, wherein said RdRp is from Hepatitis C virus.

4. The method of claim 1, wherein said RNA-dependent polymerase is an RNA-dependent DNA polymerase (RdDp).

5. The method of claim 4, wherein said RdDp is reverse transcriptase from Human Immunodeficiency virus.

6. The method of claim 1, wherein said hybridized polynucleotide comprises synthetic poly(A) and poly(U).

7. The method of claim 1, wherein said hybridized polynucleotide comprises synthetic poly(G) and poly(C).

8. The method claim 1, wherein said primer oligonucleotide and said template polynucleotide are on the same RNA molecule.

9. The method of claim 1, wherein said PPᵢ detection mixture comprises luciferase, luciferin, ATP sulphurylase and adenosine 5′-phosphosulfate (APS) and said product is emitted light.

10. The method of claim 9, wherein the emitted light is measured with a luminometer.

11. The method of claim 9, wherein said luciferase is a thermostable luciferase.

12. A method for evaluating an inhibitor of an RNA-dependent polymerase comprising:
(a) providing a primer oligonucleotide having a 3′ OH;
(b) contacting said primer oligonucleotide with a template polynucleotide and allowing hybridization to occur to form a hybridized polynucleotide;
(c) adding an RNA-dependent polymerase to said hybridized polynucleotide to produce a mixture;
(d) adding a PPᵢ detection mixture to said mixture;
(e) adding a substrate mixture comprising a nucleotide triphosphate or an analog thereof to said mixture;
(f) adding a compound that is or is suspected of being an inhibitor of said RNA-dependent polymerase; and
(g) measuring a product of the PPᵢ detection mixture;
wherein
apyrase is not part of the mixture, and
steps (c), (d), (e) and (f) may be performed simultaneously or separately in any order.

13. The method of claim 12, wherein said RNA-dependent polymerase is a viral RNA-dependent RNA polymerase (RdRp) selected from the group consisting of Hepatitis C virus, poliovirus, West Nile virus, Dengue virus, Human T Cell Leukemia virus, St. Louis Encephalitis virus, Yellow Fever virus and Measles virus.

14. The method of claim 13, wherein said RdRp is a recombinantly produced Hepatitis C virus NS5B.

15. The method of claim 12, wherein said RNA-dependent polymerase is an RNA-dependent DNA polymerase (RdDp).

16. The method of claim 15, wherein said RdDp is reverse transcriptase from Human Immunodeficiency virus.

17. The method of claim 12, wherein said hybridized polynucleotide comprises synthetic poly(A) and poly(U).

18. The method of claim 12, wherein said hybridized polynucleotide comprises synthetic poly(G) and poly(C).

19. The method claim 12, wherein said primer oligonucleotide and said template polynucleotide are on the same RNA molecule.

20. The method of claim 12, wherein said PPᵢ detection mixture comprises luciferase, luciferin, ATP sulphurylase and adenosine 5′-phosphosulfate (APS) and said product is emitted light.

21. The method of claim 20, wherein the emitted light is measured with a luminometer.

22. The method of claim 21, wherein said luciferase is a thermostable luciferase.
